(19) United States
(12) Patent Application Publication
(10) Pub. No.: US 2002/0173853 A1
(43) Pub. Date: Nov. 21, 2002

(54) Movable joint and method for coating movable joints

(76) Inventors: Harry E. Corl III, Twinsburg, OH (US); Lawrence E. Noble, Scottsdale, AZ (US)

Correspondence Address: Lawrence M. Nawrocki, NAWROCKI, ROONEY & SIVERTSON, P.A., Broadway Place East, Suite 401, 3433 Broadway Street Northeast, Minneapolis, MN, 55413, US

(21) Appl. No.: 09/859,352

(22) Filed: May 17, 2001

(51) Int. Cl. A61F 2/30

(52) U.S. Cl. 623/23.4; 623/23.41; 623/22.17

(57) ABSTRACT

A movable joint has a first portion and a second portion and at least a deposition of chromium material forming an interface between the first and second portions. The first portion has an outer surface. The second portion also has an outer surface. The deposition of chromium material forms an interface between the outer surface of the first portion and the outer surface of the second portion.

### BACKGROUND OF THE INVENTION

The present invention relates generally to the field of movable joints. In particular, the present invention involves an improved movable joint having a chromium outer surface and a method for coating the surface of a movable joint.

Movable joints have been utilized in many different technical areas, from medical implants to automobile parts, with each technical area having different, important characteristics. In some applications, the amount of constant load that a joint can maintain over a long duration is important. In other applications, the amount of extreme load that a joint can maintain over a short period of time may be important. In still other applications, the wear resistance of the joint when the parts of the joint are in relatively constant movement is important. Most applications require a mix of these important factors.

One such application is the use of a ball-type joint to replace a natural joint in a human or animal. Ball joints have proven useful in this application because, like the natural joint that the implant is replacing, the joint provides a wide range of motion. However, under these conditions, it is important to have a joint that can be in relatively constant motion and exposed to differing loads without becoming worn, thereby, requiring the joint to be replaced. Since the replacement of the joint is accomplished through invasive surgery, the longer the joint can be utilized without repair or replacement the less risk of injury from the invasive surgery or from complications therefrom.

The present invention may be utilized with any movable joint, but is particularly applicable to ball-type joints. A movable ball joint is typically comprised of two main parts; a ball portion and a socket portion. The socket is constructed to encapsulate more than half of the ball portion, thereby securing the ball portion in a movable relationship with respect to the socket.

Traditionally, the parts of these joints have been made from the same material. For example, in the field of medical implants, the most commonly utilized material has been cobalt-chromium alloys. These materials are advantageous for these uses because they are strong enough to withstand the day to day forces applied to them and they are light enough to be suitable as a replacement for the natural joint, among other suitable characteristics. However, the wear between the two parts has made the use of these devices, for long term applications, somewhat undesirable. One proposed solution has been to use different materials to construct the joint parts, wherein one material is tougher than the other material. This makes the replacement of a single part necessary instead of the replacement of both parts. However, an invasive surgery is still necessary to remove and replace the worn part and therefore, this solution still provides a substantial risk of injury to a patient. Furthermore, debris worn off of the softer part may be difficult to remove from the patient.

### SUMMARY OF THE INVENTION

The present invention offers a solution to this problem by providing a portion of the joint constructed having a chromium interface surface that reduces wear between the joint surfaces, such as both the ball and socket portions of a ball joint, by virtue of its intrinsic hardness and lubricity. The present invention generally provides a first portion and a second portion with either the first portion or the second portion having a chromium outer surface. For example, one embodiment of the present invention generally provides a ball joint, having a ball portion comprising at least a deposition of chromium forming an outer surface of the ball portion. Alternatively, the socket portion may have a deposition of chromium forming an interface surface thereon. The ball portion is adapted to be rotatably captured within a defined area of the socket portion, thereby capturing the ball portion in the socket portion. In each embodiment, the chromium deposition forms an interface surface between the first and second portions.

In a particular embodiment, the chromium material utilized for deposition on either the first or second portion of a movable joint is comprised of hexavalent chromium. The chromium material may be in the form of an electro-chemically bound, thin deposit of chromium on the outer surface of the portion. In such an embodiment, the interior structure of the portion may be comprised of a cobalt-chromium based alloy. Furthermore, the chromium may be bonded to the outer surface of the portion by electro-deposition.

In a ball-type joint, the socket portion generally has an area constructed and arranged to receive the ball portion in a movable relationship within the confines of the defined area. In one embodiment, the socket portion of the joint is formed from ultra high molecular weight polyethylene. This material provides a suitable and complimentary surface to that of a chromium deposited ball portion, thereby providing increased wear resistance to the device.

The features provided above may be combined to provide an embodiment comprising a joint having a first portion, formed of a cobalt-chromium based alloy, with an outer surface coated with a hexavalent chromium deposition applied over its outer surface, and a second portion formed from an ultra high molecular weight polyethylene material.

One application that joints, constructed according to the present invention, are particularly suited for is use in replacement of natural human or animal joints, such as knee, ankle, elbow, shoulder, spine, etc. However, the devices may be useful in any medical or non-medical application that, among other criteria, requires a joint with good wear resistance. Joints fabricated according to the present invention are also suited for these applications because they provide a reduction in fretting. Fretting is the production of wear debris through the interaction between two or more parts. The reduction of fretting reduces the chance of osteolysis, which occurs when wear debris enters the bloodstream.

One preferred method of producing a coated ball joint, comprises the steps of: providing a socket portion having an area adapted to receive a ball portion of the ball joint and the forming of either the ball or the socket portion having at least an outer interface surface comprised of chromium, wherein the ball portion is adapted to be received and captured, such that the ball portion is capable of rotatable movement, within an area of the socket portion. The method may also include the step of capturing the ball portion within the area of the socket portion. In a ball-type joint, wherein the ball is the first portion and the socket is the second portion, the socket has an area constructed and arranged to receive the ball in movable relation within the confines of the defined area and the ball portion adapted to be rotatably captured within the defined area of the socket portion.

The aforementioned benefits and other benefits including specific features of the invention will become clear from the following description by reference to the accompanying drawings.

### BRIEF DESCRIPTION OF THE DRAWINGS

FIG. 1 is a cut-away side view of a ball-type embodiment of the present invention wherein the socket has been attached to the bone surface of a patient;

FIG. 2 is a magnified cut-away side view of a portion of the ball of the implant of the embodiment of FIG. 1 showing the interface of chromium applied to the surface of the ball portion;

FIG. 3 is a cut-away side view of an embodiment of the present invention showing an interface of chromium applied to the surface of the socket portion;

FIG. 4 is a cut-away side view of the embodiment of FIG. 3; and

FIG. 5 is a cut-away side view of an embodiment of the present invention in assembled condition showing the interface of chromium applied to the surface of the ball portion.

### DETAILED DESCRIPTION OF THE INVENTION

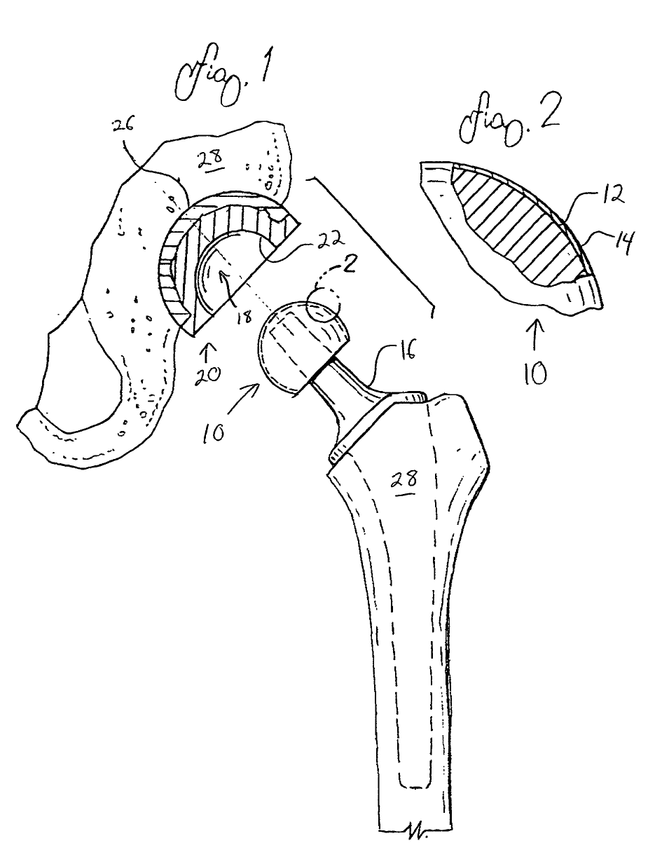

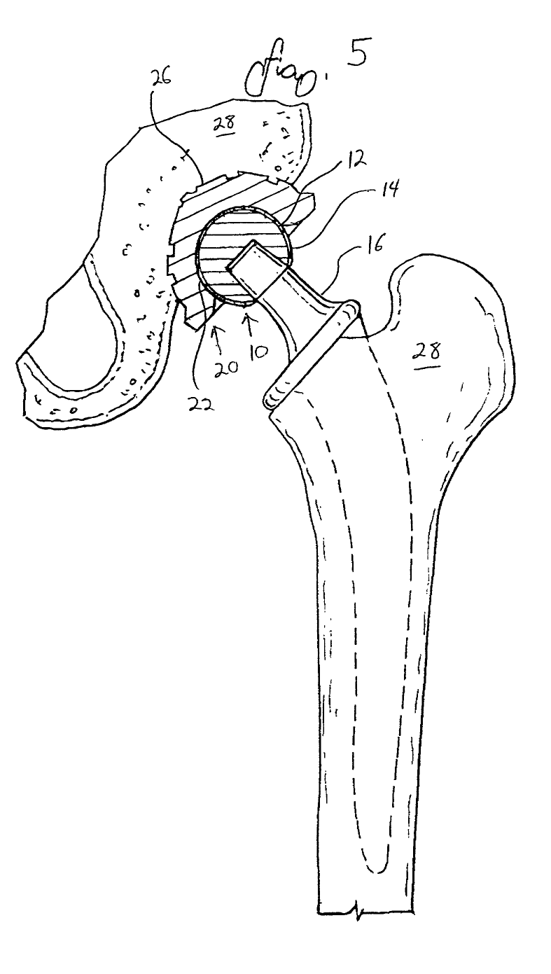

Referring now to the drawings wherein like reference numerals denote like elements throughout the several views, FIG. 1 illustrates a cut-away side view of an embodiment of the present invention. A ball-type embodiment of the present invention comprises a ball joint having a first, ball shaped, portion 10 having an outer surface 12 and a second, socket shaped, portion 20 having an outer surface 22. The ball portion 10 is sized and shaped to engage the cup 18 formed in the socket portion 20. As shown in FIGS. 4 and 5, the cup 18 is an area constructed and arranged to hold the ball portion 10 within the confines of the cup 18 and to allow the ball portion 10 to rotate within the confines of the cup 18. The ball portion 10 is typically attached to a stem 16 that is enabled to move relative to the socket portion 20 because of the rotatable engagement of the ball portion 10 with the socket portion 20.

The socket portion 20 and stem 16 of the ball portion 10 may be attached to an attachment surface 28 by any means known in the art. Some suitable examples of attachment means include: mechanical attachment assemblies, such as screws and nuts and bolts, and adhesive mechanisms, such as cement and glue, for example.

Furthermore, the shape of the surface 26 of the socket portion 20 utilized for attachment to the attachment surface 28 may be of any suitable shape known in the art. For example, FIGS. 1, 3, and 4 illustrate a socket surface 26 having a substantially uniform circular surface, whereas FIG. 5 illustrates a socket portion 20 having a non-uniform surface 26.

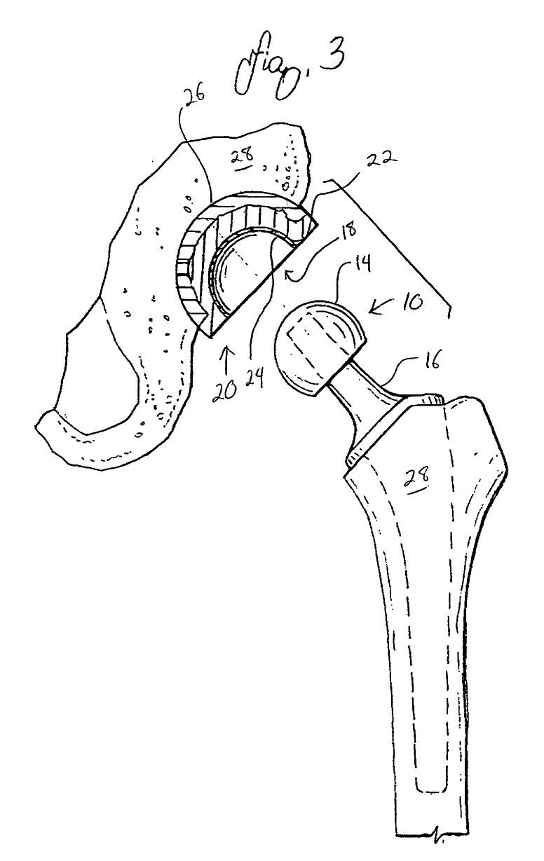

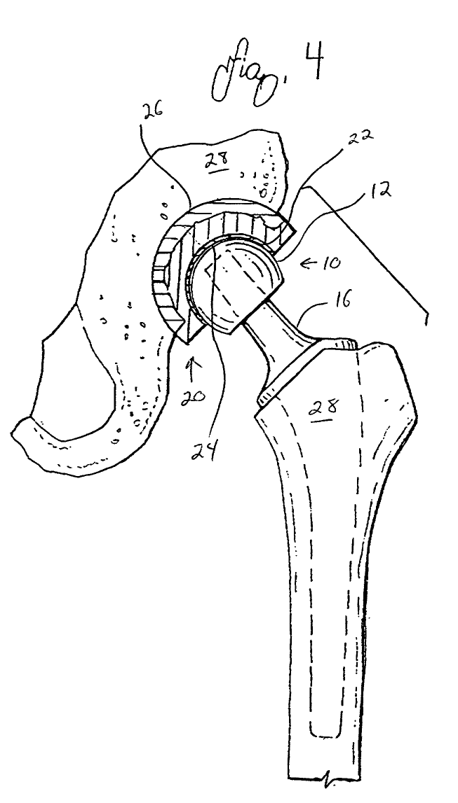

The surface coated with chromium material may be either the outer surface 12 of the first portion 10 or the outer surface 22 of the second portion 20. In the embodiment shown in FIGS. 1, 2, and 5 a thin deposition of chromium is placed over the outer surface 12 of the first portion 10. In the embodiment shown in FIGS. 3 and 4, a thin deposition of chromium is placed over the outer surface 22, generally formed within the cup 18, of the second portion 20.

By applying the chromium to one of the outer surfaces 12 or 22, the chromium provides an interface between the materials used to form the first and second portions. The interface may be utilized with any materials that form the first and second portions known in the art. For example, cobalt-chromium alloys or stainless steel are two examples of materials that may be coated with chromium within the purview of this invention.

Additionally, in a preferred embodiment of the present invention, when one of the first or second portions is coated with chromium, the other first or second portion may preferably be constructed from an ultra high molecular weight polyethylene material. For example, in one embodiment, a ball portion may be comprised of a cobalt-chromium alloy coated with a deposition of chromium and a socket portion may be constructed from ultra high molecular weight polyethylene. In another embodiment, both the first and second portions may be formed of a cobalt-chromium based alloy with one of the surfaces of the two portions having a chromium deposition thereon. As indicated above, the present invention may be provided on joints having both portions made of a single material, for example for a joint having both the first and second portions of the joint formed from metal.

It is also preferred that the chromium utilized for the deposition process be hexavalent chromium and that the deposition be electro-chemically bound. The chromium may be deposited through any process known in the art, such as electro-deposition. The deposition may occur by flash coating the surface, thereby depositing the chromium thereon. One suitable thickness for the chromium deposition is approximately 2/10,000 of an inch, however, the deposition may be as small as 50/millionths of an inch. The process of applying the coating may also include pre and post plating mechanical polishing.

Since many possible embodiments may be made of the present invention without departing from the scope thereof, it is to be understood that all matter herein set forth or shown in the accompanying drawings is to be interpreted in the illustrative and not limiting sense.

What is claimed is:

1. A movable joint, comprising;
a first portion having an outer surface; and
a second portion having an outer surface; and
at least a deposition of chromium material forming an interface between said outer surface of said first portion and said outer surface of said second portion.

2. The joint according to claim 1, wherein said depostion of chromium material is deposited on the outer surface of said first portion.

3. The joint according to claim 1, wherein said first portion is ball shaped and said second portion has an area constructed and arranged to receive said ball shaped portion in movable relation within the confines of said area and said ball shaped portion is adapted to be rotatably captured within said area of said socket portion.

4. The joint according to claim 3, wherein said area constructed and arranged to receive said ball shaped portion has an outer surface and wherein said deposition of chromium material is deposited on the outer surface of said area of said socket portion.

5. The joint according to claim 1, wherein said chromium material is hexavalent chromium.

6. The joint according to claim 1, wherein said second portion of said joint is formed from ultra high molecular weight polyethylene.

7. The joint according to claim 1, wherein said chromium deposition is in the form of an electro-chemically bound, thin deposit of chromium on said outer surface of said first portion.

8. The joint according to claim 1, wherein said chromium is bonded to the said outer surface of said first portion by electro-deposition.

9. The joint according to claim 1, wherein said first portion is constructed from cobalt-chromium based alloy.

10. The joint according to claim 1, wherein said first and second portions are constructed from cobalt-chromium based alloy.

11. The joint according to claim 1, wherein said movable joint is adapted to replace a joint in the human body.

12. The joint according to claim 1, wherein said movable joint is a knee joint implant.

13. The joint according to claim 3, wherein said socket portion of said joint is formed from ultra high molecular weight polyethylene.

14. The joint according to claim 13, wherein said chromium deposition is in the form of an electro-chemically bound, thin deposit of chromium on said outer surface of said ball portion.

15. The joint according to claim 14, wherein said coating is bonded to said outer surface of said ball portion by electro-deposition.

16. The joint according to claim 14, wherein said ball portion is constructed from cobalt-chromium based alloy with a chromium deposition formed on its outer surface.

17. A method of producing a coated movable joint, the steps of the method, comprising;
providing a first portion having an outer surface and a second portion with an area adapted to interface with a portion of the outer surface of said first portion of said joint thereby forming an interface between said first portion and said second portion such that said first and second portions are capable of movement with respect to each other; and
providing a deposition of chromium material at said interface between said first portion and said second portion.

18. The method according to claim 17, wherein said step of providing a deposition of chromium material is provided by depositing said material on the outer surface of said ball portion.

19. The method according to claim 17, wherein said area constructed and arranged to receive said ball portion has an outer surface and wherein said step of providing a deposition of chromium material is provided by depositing said material on the outer surface of said area of said socket portion.

20. The method according to claim 17, wherein said chromium material is hexavalent chromium.

21. The method according to claim 17, wherein said socket portion of said joint is formed from ultra high molecular weight polyethylene.

22. The method according to claim 17, wherein said chromium deposition is in the form of an electro-chemically bound, thin deposit of chromium on said outer surface of said ball portion.

23. The method according to claim 17, wherein said chromium is bonded to said outer surface by electro-deposition.

24. A movable joint, comprising;
a first portion, formed of a cobalt-chromium based alloy, having an outer surface coated with a hexavalent chromium deposition applied over said outer surface; and
a second portion, formed of an ultra high molecular weight polyethylene material, having an area constructed and arranged to interface with said first portion such that said first and second portions are in movable relation with respect to each other.

25. A movable joint, comprising;
a first portion, formed of an ultra high molecular weight polyethylene material, and
a second portion, formed of a cobalt-chromium based alloy, having an outer surface coated with a hexavalent chromium deposition applied over said outer surface, having an area constructed and arranged to interface with said first portion such that said first and second portions are in movable relation with respect to each other.
